(19) United States
(12) Patent Application Publication
(10) Pub. No.: US 2026/0174821 A1
(43) Pub. Date: Jun. 25, 2026

(54) COMPOSITIONS AND METHODS FOR TISSUE REPAIR

(71) Applicant: Arizona Board of Regents on Behalf of the University of Arizona, Tucson, AZ (US)

(72) Inventors: Steven Goldman, Tucson, AZ (US); Jennifer Koevary, Tucson, AZ (US); Jordan Lancaster, Tucson, AZ (US); Jordan Fink, Tucson, AZ (US); Adrian Grijalva, Tucson, AZ (US)

(21) Appl. No.: 19/124,914

(22) PCT Filed: Oct. 30, 2023
(86) PCT No.: PCT/US2023/036279

§ 371 Date: Apr. 28, 2025

Related U.S. Application Data
(60) Provisional application No. 63/420,250, filed on Oct. 28, 2022.

(51) Int. Cl. A61K 38/00; A61K 9/00; A61K 35/28; A61K 45/06

(52) U.S. Cl. CPC A61K 38/00; A61K 9/0019; A61K 35/28; A61K 45/06

(57) ABSTRACT

Provided herein are compositions and methods for tissue repair. In particular, the compositions and methods described herein find use in repairing, regenerating, and modulating fibrosis and inflammation in a variety of tissues such as, for example, large volume muscle loss, including skeletal and heart muscle, blast wave injury, muscle loss from diabetes and resulting from frailty as a result of aging.

### CROSS-REFERENCE TO RELATED APPLICATION

This application claims priority to U.S. provisional patent application Ser. No. 63/420,250, filed Oct. 28, 2022, which is incorporated herein by reference in its entirety.

### FIELD

Provided herein are compositions and methods for tissue repair. In particular, the compositions and methods described herein find use in repairing and modulating fibrosis and inflammation (e.g., in tissues) to treat a variety of diseases and conditions.

### BACKGROUND

Fibrosis, also known as fibrotic scarring, is a pathological wound healing in which connective tissue replaces normal parenchymal tissue to the extent that it can go unchecked, leading to considerable tissue remodeling and the formation of permanent scar tissue.

Repeated injuries, chronic inflammation and repair are susceptible to fibrosis, where an accidental excessive accumulation of extracellular matrix components, such as the collagen, is produced by fibroblasts, leading to the formation of a permanent fibrotic scar.

In response to injury, this is called scarring, and if fibrosis arises from a single cell line, this is a fibroma. Physiologically, fibrosis acts to deposit connective tissue, which can interfere with or totally inhibit the normal architecture and function of the underlying organ or tissue. Fibrosis can be used to describe the pathological state of excess deposition of fibrous tissue, as well as the process of connective tissue deposition in healing. Defined by the pathological accumulation of extracellular matrix (ECM) proteins, fibrosis results in scarring and thickening of the affected tissue—it is in essence an exaggerated wound healing response which can interfere with normal organ function.

For example, chronic heart failure (CHF) is one of the leading causes of death in the United States and commonly includes fibrosis in the heart muscle of varying severity. Common causes of heart failure include coronary artery disease including a previous myocardial infarction (heart attack), high blood pressure, atrial fibrillation, valvular heart disease, excess alcohol use, infection, cardiomyopathy of an unknown cause and several genetic diseases. These cause heart failure by changing either the structure or the functioning of the heart, including fibrotic changes. The two types of heart failure-heart failure with reduced ejection fraction (HFrEF), and heart failure with preserved ejection fraction (HFpEF)—are based on whether the ability of the left ventricle to contract and relax is affected. The severity of disease is graded by the severity of symptoms with at rest and with exercise. Heart failure is not the same as myocardial infarction (in which part of the heart muscle dies) or cardiac arrest (in which blood flow stops altogether). Heart failure is diagnosed based on the history of the symptoms and a physical examination, with confirmation by echocardiography, magnetic resonance imaging, and computed tomography. Blood tests, electrocardiogra and chest radiography may be useful to determine the underlying cause.

Treatment depends on the severity and cause of the disease. In people with chronic stable mild heart failure, treatment commonly consists of lifestyle modifications such as stopping smoking, physical exercise, and dietary changes, as well as medications. In those with heart failure due to left ventricular dysfunction, angiotensin converting enzyme inhibitors, angiotensin receptor blockers, or valsartan/sacubitril and sodium-glucose co-transporter 2 (SGLT-2) inhibitors along with beta blockers are recommended. For those with severe disease, aldosterone antagonists, or hydralazine with a nitrate may be used. Diuretics are useful for preventing fluid retention. Sometimes, depending on the cause, an implanted device such as a pacemaker or an implantable cardiac defibrillator (ICD) may be recommended. In some moderate or severe cases, cardiac resynchronization therapy (CRT) or cardiac contractility modulation may be of benefit. A ventricular assist device, an artificial heart or occasionally a heart transplant may be recommended in those with severe disease that persists despite all other measures.

There is a need for additional treatments for tissue damage in diseases and conditions such as CHF that address the primary cause of the disease, i.e., loss of functioning heart cells or cardiomyocytes.

The present invention addresses this need.

### SUMMARY

Experiments described herein identified host immune system factors including genes involved in tissue (e.g., muscle) repair and/or regeneration. Accordingly, described herein are compositions that promote tissue regeneration and/or repair by inducing expression of factors (e.g., genes) associated with tissue regeneration and/or repair (e.g., via immune modulation) or by providing polypeptides associated with tissue regeneration and/or repair.

For example, in some embodiments, provided is a method of regenerating and/or repairing a tissue, comprising contacting a tissue with a composition comprising at least a portion of one or more polypeptides and/or proteins selected from those shown in Table 2.

Further embodiments provide a method of regenerating and/or repairing a tissue, comprising contacting the tissue with a composition comprising a nucleic acid encoding at least a portion of one or more polypeptides and/or proteins selected from those shown in Table 2.

Additional embodiments provide a method of regenerating and/or repairing a tissue, comprising contacting the tissue with a composition that induces or decreases expression of genes associated with tissue repair and/or regeneration of the tissue.

In some embodiments, the composition induces differential expression of genes involved in host innate and adaptive immune system and/or induces recruitment/activation of M2 macrophages, M1 macrohages, and/or stromal cells. Examples include but are not limited to RETNLA, ARG1, CHIL3, FN1, or MRC1. In some embodiments, the composition comprises cells that express one or more exogenous genes selected from RETNLA, ARG1, CHIL3, FN1, or MRC1.

In some embodiments, the composition decreases expression of one or more genes selected from those shown in Table 1 and increases expression of one or more genes shown in Table 2.

In some embodiments, the composition regulates extracellular matrix (ECM) organization. In some embodiments, the composition promotes signaling by one or more molecules selected from IL-4, IGF, or IL-13.

The present disclosure is not limited to a particular disease, condition or disorder. Non limiting examples include but are not limited to fibrosis (e.g., lung or liver fibrosis), blast wave injury, muscle loss, skin damage, CHF, aging, etc. The present disclosure is not limited to a particular tissue. Non limiting examples include but are not limited to, bone marrow, brain, skin, lung, liver, heart, kidney, etc. In some exemplary embodiments, the tissue is a muscle (e.g., heart or skeletal muscle, and the like).

In some embodiments, the composition reduces or increases fibrosis and/or inflammation in the tissue. In some embodiments, the composition changes the makeup of the immune cells from a fibrotic and inflammatory type response to a reparative type repose, or vice-versa.

In some embodiments, the composition comprises a solid or semi-solid support (e.g., including but not limited to a scaffold, a gel, a nanoparticle, or a patch). In some embodiments, the polypeptide, protein, or nucleic acid is affixed to the solid or semi-solid support. In some embodiments, the composition comprises a pharmaceutically acceptable carrier. In some embodiments, the composition comprises a virus-like particle encoding the one or more polypeptides and/or proteins. In some embodiments, the composition is delivered with a metered dose inhaler.

Also provided is a composition, comprising a solid or semi solid support comprising one or more polypeptides and/or proteins selected from those shown in Table 2.

Additional embodiments are described herein.

### BRIEF DESCRIPTION OF THE DRAWINGS

FIG. 1 shows a schematic representation of how Digital; Spatial Profiling (DSP) experiments are done showing the time sequence from creating the myocardial infarction (MI) in the mouse to performing the DSP analysis.

FIG. 2 shows the infarcted heart in the mouse, showing that with immunohistochemistry one can define the specific cell types in different areas of the heart: with scar after MI (A) and scar area after placement of the patch (B).

FIG. 3 shows a schematic of the incidence of heart failure.

FIG. 4 shows an exemplary cardiac patch.

FIG. 5 shows a patch treated rat (left) and swine (right).

FIG. 6 shows an immuno histochemical (IHC) stain of 1 week post treatment heart.

FIG. 7 shows region of intertest (ROI) selection of control CHF.

FIG. 8 shows ROI in patch treated mice.

FIG. 9 shows the degree of MI vs. MI treated with a patch.

FIG. 10 shows immune cell diversity after patch treatment.

FIG. 11 shows elevated DC and macrophages after patch treatment.

FIG. 12 shows IHC after patch treatment.

FIG. 13 shows ROI in CHF mice.

FIG. 14 shows ROI in patch treated mice.

FIG. 15 shows tissue similarity after patch treatment.

FIG. 16 shows CD45 segments of MI and patch treatment tissue.

FIG. 17 shows region dependent expression of macrophage markers.

FIG. 18 shows study design for exemplary experiments.

FIG. 19A-C shows A) Immunofluorescence image from the digital spatial profiling experiment showing the type sections transcriptome data was obtained from. 1) Border zone, 2) Infarct zone, and 3) patch. B) Gene expression data from the CD45 and unlabeled cell sections showing regenerative paracrine factor expression is much higher in MI-Patch_Patch C) Gene expression data showing macrophage expression in the CD45 and unlabeled cell groups is increased in MI-Patch_Patch.

FIG. 20A-C shows a PCA analysis of immune regions vs timepoint with PC1 accounting for 68% variation after patch placement.

FIG. 21 shows a temporal analysis of ECM signaling after patch placement.

FIG. 22 shows a timepoint of collagen expression after patch placement.

FIG. 23 shows a timepoint of Tenascin C (TNC) expression after patch placement.

FIG. 24 shows a schematic of proteomics experiments during patch production.

FIG. 25 shows a heatmap of proteomics expression profiles.

FIG. 26 shows signaling enriched Reactome pathways and Gene ontology terms identified from the proteomics data.

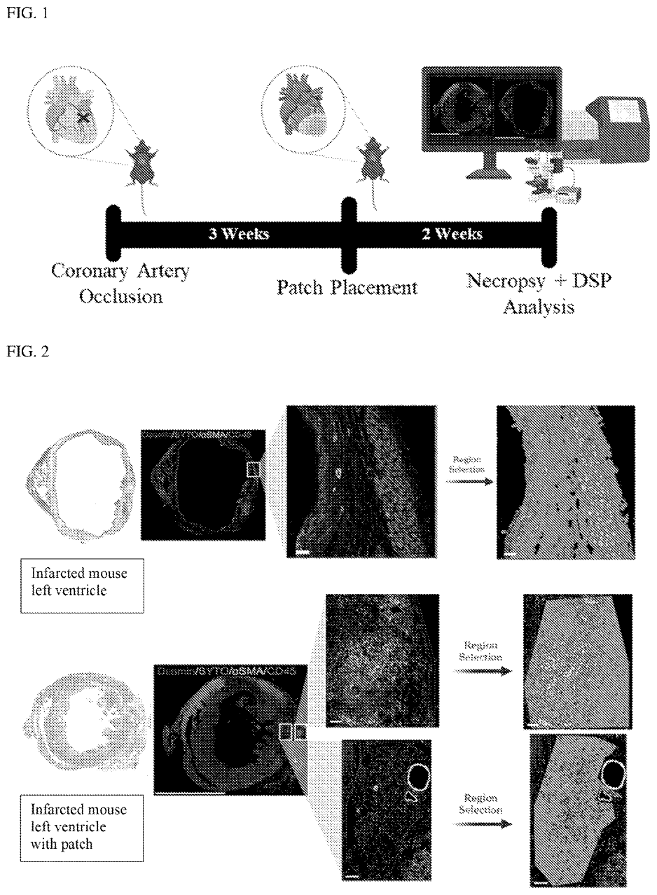

### DESCRIPTION

Provided herein are compositions and methods for tissue repair. In particular, the compositions and methods described herein find use in repairing, regenerating, and modulating fibrosis and inflammation in a variety of tissues such as, for example, skeletal muscle, lung, bone, cartilage, liver, skin and heart muscle.

While the present disclosure is exemplified with CHF and skeletal muscle repair, the present invention is not limited to treatment of a particular tissue or disease. Non limiting examples include but are not limited to fibrosis (e.g., lung or liver fibrosis), blast wave injury, CHF, aging, muscle loss (e.g., from diabetes or aging), organ damage, etc. The present disclosure is not limited to a particular tissue. Non limiting examples include but are not limited to, bone marrow, brain, skin, lung, liver, heart, kidney, etc. In some exemplary embodiments, the tissue is a muscle (e.g., heart or skeletal muscle, and the like).

Digital Spatial Profiling (DSP) is a technique that takes two concepts of immunohistochemistry (IHC) and Next Generation Sequencing (NGS) and merges them to provide ribonucleic acid (RNA) and protein transcriptomics within a certain region of interest on a sample of tissue. Using morphology antibodies to provide highlighted cell types, the GeoMx instrument (nanoString® Inc) uses a combination of ultraviolet (UV) light and mirrors to extract these cells via oligo tags, e.g., short single strands of synthetic DNA or RNA that serve as the starting point for many molecular biology and synthetic biology application. Once these tags are lifted, they are sipped up by the GeoMx and transferred to an Illumina plate to run next gen sequencing. The GeoMx instrument associates the acquired transcriptomic data with the region of interest in the sample. The result is biologically spatially relevant data allowing investigation of gene pathways altered in that specific moment of time as well as specific interactions between cell types isolated. To-date, DSP has been mostly used to develop personalized medical treatments for cancer, where investigators biopsy a cancer, define the changes in gene expression at the site of the cancer and develop specific treatments for that specific patient to treat the changes in gene activation caused by that cancer.

In experiments described herein DSP was used to examine the acute and chronic transcriptional changes of a regenerative therapy-namely a biodegradable patch seeded with human neonatal dermal fibroblasts and human induced pluripotent stem cell derived cardiomyocytes (iPSC-CMs). Results identified genes associated with tissue repair and regeneration (See e.g., Lancaster J J, Grijalva A, Fink J, Ref J, Daugherty S, Whitman S, Fox K, Gorman G, Lancaster L D Avery R, Acharya T, McArthur A, Strom J, Pierce M K, Moukabary T, Borgstrom M, Benson D, Mangiola M, Pandey A C, Zile M R, Bradshaw A, Koevary J W, Goldman S. Biologically Derived Epicardial Patch Induces Macrophage Mediated Pathophysiologic Repair in Chronically Infarcted Swine Hearts Nature Biologic Communications).

Further experiments utilized proteomics approaches to identify specific pathways and proteins involved in immune responses such as inflammation and fibrosis in tissue repair and regeneration. These experiments demonstrated that patch manufacture is enriched for anti-inflammatory mechanism via regulation of ECM organization combined with M2 polarization signaled by IL-4 and IL-13. GO terms enriched for biological processes increase wound healing mechanisms, angiogenesis, and IGF signaling. These mechanisms are induced by the biologic via secretion of paracrine molecules.

The present invention thus provides a tunable immunomodulatory therapy for treatment of a variety of conditions related to inflammation and aging such as tissue repair and/or regeneration. The identification of specific proteins involved in such processes allows for the tuning (e.g., selectively increasing or decreasing) of inflammatory and fibrotic responses to address specific disease and conditions. For example, embodiments of the present invention provide for customized up or down regulation of specific genes involved in an immune response to customize treatment for a specific disease or condition.

Accordingly, provided herein are compositions and methods for tissue (e.g., cardiac and other muscle tissue) regeneration and/or repair. Leveraging the changes of genetic expression induced by a composition described herein allows one to create target pathways that can increase regeneration and replace scar tissue with new functioning cells. For example, in some embodiments, a patch that comprises genetically engineered fibroblast that promote M2 and/or M1 macrophage activation and infiltration is provided. Increasing activation of M2 macrophages increases the repair of endogenous cells and decrease the amount of fibrosis in the damaged tissue. In some embodiments provided is an engineered cell that induces macrophage polarization to M2 and results in up regulation of genes such as, for example, one or more of RETNLA, ARG1, CHIL3, FN1, and MRC1 or induces macrophage polarization to M1. In some embodiments, modulation of at least a portion (e.g., from 5, 10, 20, or 30 amino acids to the entire) proteins or polypeptides shown in Tables 1 and 2 is provided by providing protein and/or genetic therapies.

The changes in gene expression are not specific to cardiac repair. For example, in some embodiments, other cell or tissue regeneration and/or repair pathways are induced. Examples include but are not limited to treating skeletal muscle loss by promoting repair of endogenous skeletal muscle cells. By weight, skeletal muscle is the most abundant tissue in the body, accounting for ˜40% of our total body weight. After injury, skeletal muscle grows back in limited quantities, resulting in permanent disability reducing quality of life and increasing pain. Prior to the present disclosure, the current state of the art held that the repair or regeneration of damaged tissues such as cardiac or skeletal muscle is limited due to the poor regenerative capacity of these tissues. However, as demonstrated herein muscle regeneration does occur. The economic impact of skeletal muscle/soft tissue repair is large. “Soft Tissue Repair Market Size” has been valued at over $10.2 billion in 2021 and is estimated to have combine annual growth rate between 2022 and 2030 of more than 7.8% (gminsights.com/industry-analysis/soft-tissue-repair-market).

In some embodiments, the treatments described herein reduce fibrosis and/or inflammation in a tissue.

Further embodiments provide gene therapy to target cell and organ loss, with or without scaffolds described herein. For example, in some embodiments, specific gene therapy to induce regeneration is targeted into contained areas of the body like the spinal column, to regenerate nerves and treat patients with spinal cord injury and other applications.

In some embodiments, polypeptides and/or proteins that promote tissue repair and/or regeneration are delivered via autologous hematopoietic stem cell (HSC) gene therapy using virus like particles (e.g., capsid-modified, helper-dependent HDAd5/35++ vectors (Chang Li, et al., JCI Insight. 2022; 7 (19): e162939; herein incorporated by reference in its entirety)).

In some embodiments, compositions and methods of the present disclosure utilize solid or semi solid supports (e.g., scaffold, a gel, a nanoparticle, or a patch) to deliver a therapeutic.

In some embodiments, the solid support is a scaffold. In some embodiments, scaffolds comprise a biomaterial support comprising fibroblasts and/or cardiomyocytes.

The scaffolds described herein are scaffolded of any number of suitable materials. In some embodiments, the scaffold comprises synthetic material. In some embodiments, the scaffold comprises biological material. In some embodiments, the scaffold is a hybrid of synthetic and biological materials.

Examples of suitable scaffold material include, but are not limited to, one or more of collagen, fibronectin, polyglycolides, polylactides, polypropylene, polyester, silicone, expanded polytetrafluorothylene, Dexon, Vicryl, polycaprolactone, polydioxanone, catgut, silk, nylon, and trimethylene carbonate.

For certain application, the scaffold is composed of a polylactide material or a polyglactin 910 material. In some embodiments, the scaffold is derived from human, bovine or porcine tissue.

In some embodiments, the scaffold is bioabsorbable. In some embodiments, the scaffold is non-bioabsorbable.

For certain applications, the scaffold further comprises a therapeutic agent such as a drug or biologic. In some embodiments, the therapeutic agent is known to be useful in treating, ameliorating and/or preventing cardiac conditions. Examples include, but are not limited to, angiotensin-converting enzyme (ACE) inhibitors (e.g., enalapril, lisinopril, and captopril), angiotensin II (A-II) receptor blockers (e.g., losartan and valsartan), diuretics (e.g., bumetanide, furosemide, and spironolactone), beta blockers, nesiritide, and SGLT2 inhibitors.

In some embodiments, the scaffold further comprises cells (e.g., of the same or mixed cell types). In some embodiments, the cells are stem cells (e.g., cardiac stems cells or progenitors thereof) or derived from stem cells or fibroblasts.

In some exemplary embodiments, the compositions and methods described herein treat chronic heart failure (CHF). As used herein, “CHF” is a chronic (as opposed to rapid onset) impairment of the heart's ability to supply adequate blood to meet the body's needs. CHF may be caused by, but is distinct from, cardiac arrest, myocardial infarction, and cardiomyopathy. In one alternative embodiment, the subject suffers from congestive heart failure. In various further alternative embodiments that can be combined with any other embodiments herein, the subject's heart failure comprises left heart failure, right heart failure, backward heart failure (increased venous back pressure), forward heart failure (failure to supply adequate arterial perfusion), systolic dysfunction, diastolic dysfunction, systemic vascular resistance, low-output heart failure, high-output heart failure. In various further alternative embodiments that can be combined with any other embodiments herein, the subject's CHF may be any of Classes I-IV as per the New York Heart Association Functional Classification; more preferably Class III or IV.
Class I: no limitation is experienced in any activities; there are no symptoms from ordinary activities.
Class II: slight, mild limitation of activity; the patient is comfortable at rest or with mild exertion.
Class III: marked limitation of any activity; the patient is comfortable only at rest.
Class IV: any physical activity brings on discomfort and symptoms occur at rest.

In a further alternative embodiment that can be combined with any other embodiments herein, the subject has been diagnosed with CHF according to the New York Heart Association Functional Classification. In a further alternative embodiment that can be combined with any other embodiments herein, the subject is further characterized by one or more of the following: hypertension, obesity, cigarette smoking, diabetes, valvular heart disease, and ischemic heart disease.

As used herein, “treat” or “treating” means accomplishing one or more of the following: (a) reducing the severity of the disorder (ex: treatment of Class IV subject to improve status to Class III for CHF subjects); (b) limiting or preventing development of symptoms characteristic of the disorder; (c) inhibiting worsening of symptoms characteristic of the disorder; (d) limiting or preventing recurrence of symptoms in patients that were previously symptomatic for the disorder; and (e) increasing life span (e.g., improving mortality). Signs characteristic of CHF include, but are not limited to reduced ejection fraction, reduced myocardial perfusion, maladaptive cardiac remodeling (such as left ventricle remodeling), reduced left ventricle function, dyspnea on exertion, dyspnea at rest, orthopnea, tachypnea, paroxysmal nocturnal dyspnea, dizziness, confusion, cool extremities at rest, exercise intolerance, easy fatigability, peripheral edema, nocturia, ascites, hepatomegaly, pulmonary edema, cyanosis, laterally displaced apex beat, gallop rhythm, heart murmurs, parasternal heave, and pleural effusion.

In some embodiments, the treating comprises one or more of improving right ventricular function, improving left ventricular function, fall in left ventricular end diastolic pressure (EDP), improving myocardial perfusion, repopulating of the heart's wall with functioning previously hibernating cardiomyocytes, and reversing maladaptive left ventricle remodeling in CHF subjects.

The scaffold can be contacted with the heart or other muscle in any suitable way to promote attachment. The scaffold may be attached to various locations on the heart, including the epicardium, myocardium and endocardium, most preferably the epicardium. Means for attachment include, but are not limited to, direct adherence between the scaffold and the heart tissue, biological glue, suture, synthetic glue, laser dyes, or hydrogel. A number of commercially available hemostatic agents and sealants include SURGICAL® (oxidized cellulose), ACTIFOAM® (collagen), FIBRX® (light-activated fibrin sealant), BOHEAL® (fibrin sealant), FIBROCAPS® (dry powder fibrin sealant), polysaccharide polymers p-GlcNAc (SYVEC® patch; Marine Polymer Technologies), Polymer 27CK (Protein Polymer Tech.). Medical devices and apparatus for preparing autologous fibrin sealants from 120 ml of a patient's blood in the operating room in one and one-half hour are also known (e.g. Vivostat System).

In an alternative embodiment of the invention utilizing direct adherence, the scaffold is placed directly onto the muscle and the product attaches via natural cellular attachment. In a further alternative embodiment, the scaffold is attached to the heart using surgical glue, preferably biological glue such as a fibrin glue. The use of fibrin glue as a surgical adhesive is well known. Fibrin glue compositions are known (e.g., see U.S. Pat. Nos. 4,414,971; 4,627,879 and 5,290,552) and the derived fibrin may be autologous (e.g., see U.S. Pat. No. 5,643,192). The glue compositions may also include additional components, such as liposomes containing one or more agent or drug (e.g., see U.S. Pat. Nos. 4,359,049 and 5,605,541) and include via injection (e.g., see U.S. Pat. No. 4,874,368) or by spraying (e.g., see U.S. Pat. Nos. 5,368,563 and 5,759,171). Kits are also available for applying fibrin glue compositions (e.g., see U.S. Pat. No. 5,318,524).

In another embodiment, a laser dye is applied to the muscle, the scaffold, or both, and activated using a laser of the appropriate wavelength to adhere to the tissues. In alternative embodiments, the laser dye has an activation frequency in a range that does not alter tissue function or integrity. For instance, 800 nm light passes through tissues and red blood cells. Using indocyan green (ICG) as the laser dye, laser wavelengths that pass through tissue may be used. A solution of 5 mg/ml of ICG is painted onto the surface of the three-dimensional stromal tissue (or target site) and the ICG binds to the collagen of the tissue. A 5 ms pulse from a laser emitting light with a peak intensity near 800 nm is used to activate the laser dye, resulting in the denaturation of collagen which fuses elastin of the adjacent tissue to the modified surface.

In another embodiment, the scaffold is attached to the muscle using a hydrogel. A number of natural and synthetic polymeric materials are sufficient for forming suitable hydrogel compositions. For example, polysaccharides, e.g., alginate, may be crosslinked with divalent cations, polyphosphazenes and polyacrylates are crosslinked ionically or by ultraviolet polymerization (U.S. Pat. No. 5,709,854). Alternatively, a synthetic surgical glue such as 2-octyl cyanoacrylate (“DERMABOND™”, Ethicon, Inc., Somerville, N.J.) may be used to attach the three-dimensional stromal tissue.

In an alternative embodiment of the present invention, the scaffold is secured to the muscle using one or more sutures, including, but not limited to, 5-O, 6-O and 7-O proline sutures (Ethicon Cat. Nos. 8713H, 8714H and 8701H), poliglecaprone, polydioxanone, polyglactin or other suitable non-biodegradable or biodegradable suture material. When suturing, double armed needles are typically, although not necessarily, used.

The methods and compositions described herein can be used in combination with conventional treatments, such as the administration of various pharmaceutical agents and surgical procedures. Medications suitable for use in the methods described herein include angiotensin-converting enzyme (ACE) inhibitors (e.g., enalapril, lisinopril, and captopril), angiotensin II (A-II) receptor blockers (e.g., losartan and valsartan), diuretics (e.g., bumetanide, furosemide, and spironolactone), beta blockers, nesiritide, and SGLT2 inhibitors.

A number of methods can be used to measure changes in the functioning of the heart in subjects before and after attachment of the scaffold. For example, an echocardiogram can be used to determine the capacity at which the heart is pumping. The percentage of blood pumped out of the left ventricle, i.e., the left ventricular stroke volume divided by the left ventricular end-diastolic volume with each heartbeat is referred to as the ejection fraction. In a healthy heart, the ejection fraction is about 60 percent. In an individual with chronic heart failure caused by the inability of the left ventricle to contract vigorously, i.e., systolic heart failure, the ejection fraction is usually less than 40 percent. Depending on the severity and cause of the heart failure, ejection fractions typically range from less than 40 percent to 15 percent or less. An echocardiogram or magnetic resonance imaging can also be used to distinguish between systolic heart failure and diastolic heart failure, in which the pumping function is normal but the heart is stiff.

In some embodiments, echocardiograms or magnetic resonance images are used to compare the ejection fractions and structural changes in the heart before and following treatment with the scaffold.

Nuclear scans, such as radionuclide ventriculography (RNV) or multiple gated acquisition (MUGA) scanning can be used to determine how much blood the heart pumps with each beat. These tests are done using a small amount of dye injected in the veins of an individual A special camera is used to detect the radioactive material as it flows through the heart. Other tests include X-rays, MRI, and blood tests. Chest X-rays can be used to determine the size of the heart and if fluid has accumulated in the lungs. Blood tests can be used to check for a specific indicator of congestive heart failure, brain natriuretic peptide (BNP). BNP is secreted by the heart in high levels when it is overworked. Thus, changes in the level of BNP in the blood can be used to monitor the efficacy of the treatment regime.

In some embodiments, the compositions described herein find use in the treatment of fibrosis (e.g., lung or liver fibrosis). For example, in some embodiments, the compositions find use in treating pulmonary fibrosis. Pulmonary fibrosis is a condition in which the lungs become scarred over time. Symptoms include shortness of breath, a dry cough, feeling tired, weight loss, and nail clubbing. Complications may include pulmonary hypertension, respiratory failure, pneumothorax, and lung cancer. No cure exists and only limited treatment options are available. Treatment is directed towards efforts to improve symptoms and may include oxygen therapy and pulmonary rehabilitation. Certain medications may be used to try to slow the worsening of scarring. Lung transplantation may occasionally be an option. At least 5 million people are affected globally. Life expectancy is generally less than five years.

Certain aspects of the invention find use in the treatment of blast wave injury (e.g., by modulating the immune response to a blast exposure). A blast injury is a complex type of physical trauma resulting from direct or indirect exposure to an explosion. Blast injuries occur with the detonation of high-order explosives as well as the deflagration of low order explosives. These injuries are compounded when the explosion occurs in a confined space. Blast injuries can cause hidden sensory, bone marrow, and brain damage, with potential neurological and neurosensory consequences. Individuals exposed to blast frequently manifest loss of memory of events before and after explosion, confusion, headache, impaired sense of reality, and reduced decision-making ability. Patients with brain injuries acquired in explosions often develop sudden, unexpected brain swelling and cerebral vasospasm despite continuous monitoring. However, the first symptoms of blast-induced neurotrauma (BINT) may occur months or even years after the initial event, and are therefore categorized as secondary brain injuries. The broad variety of symptoms includes weight loss, hormone imbalance, chronic fatigue, headache, and problems in memory, speech and balance. These changes are often debilitating, interfering with daily activities. Because BINT in blast victims is underestimated, valuable time is often lost for preventive therapy and/or timely rehabilitation. Blast Wave injuries are thought to be mediated through immune system dysfuction, thus setting up an ideal mileu for the compositions and method of the present disclosure of modulating the immune system response (Bergmann-Leitner E S, Bobrov A G, Bolton J S, et al., Blast Waves Cause Immune System Dysfunction and Transient Bone Marrow Failure in a Mouse Model. Front Bioeng Biotechnol. 2022 Mar. 22; 10:821169. doi: 10.3389/fbioe.2022.821169. PMID: 35392409; PMCID: PMC8980552).

In a further aspect, the present invention provides kits for treating disorder (e.g., CHF), comprising a composition as disclosed above and a means for delivering the composition (e.g., a solid or semi-solid support comprising one or more proteins and/or polypeptides).

### EXPERIMENTAL

The following examples are provided to demonstrate and further illustrate certain embodiments of the present disclosure and are not to be construed as limiting the scope thereof.

### Example 1

DSP analysis was performed in a mouse model of ischemic CHF. A mouse is infarcted by ligating the left coronary artery. After waiting 3 weeks for the mouse to develop CHF, the patch is implanted. After another 2 weeks, the DSP analysis is performed.

The DSP analyses define the specific changes in gene expression at the site of injury and repair with the patch. The patch creates an immune response that recruits macrophages and dendritic cells into the infarcted heart. This immune response is defined with infiltration of tissue resident (TR) macrophages and hematopoietic derived M2 macrophages that secrete IGF1, IL-4, IL-13, Fstl1, and Metrnl, this is absent in non-treated animals. The immune response instigated by the iPSC-CMs and fibroblasts, namely the recruited macrophages, are important for long-term cardiac regeneration. The cellular mechanism of endogenous regeneration stems from coactivation of cell proliferative and angiogenic pathways via signaling of various immune cells that reveal M2 specific markers via spatial transcriptomic profiling in the infarcted and patch regions. Bioinformatics is used to define the specific pathways activated in cardiac regeneration.

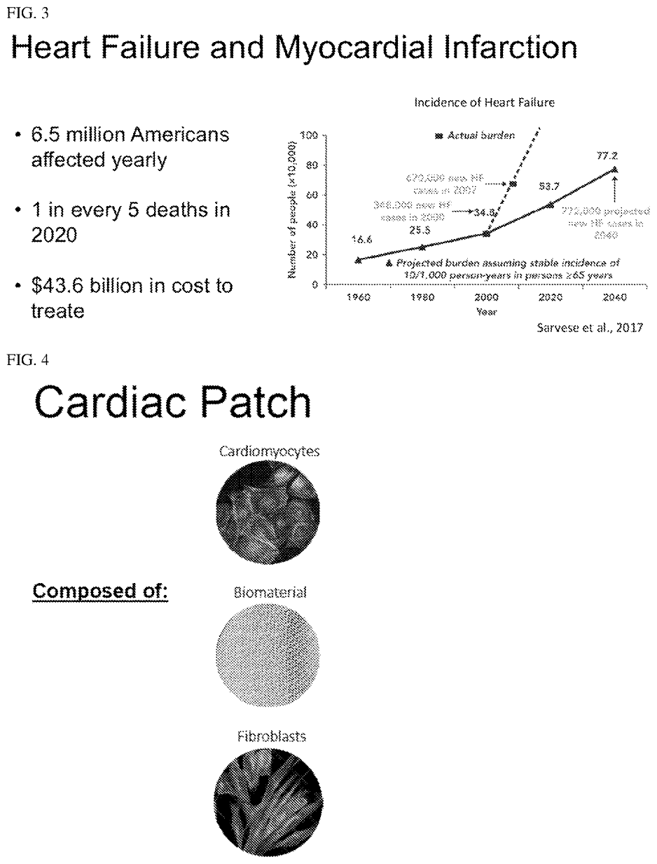

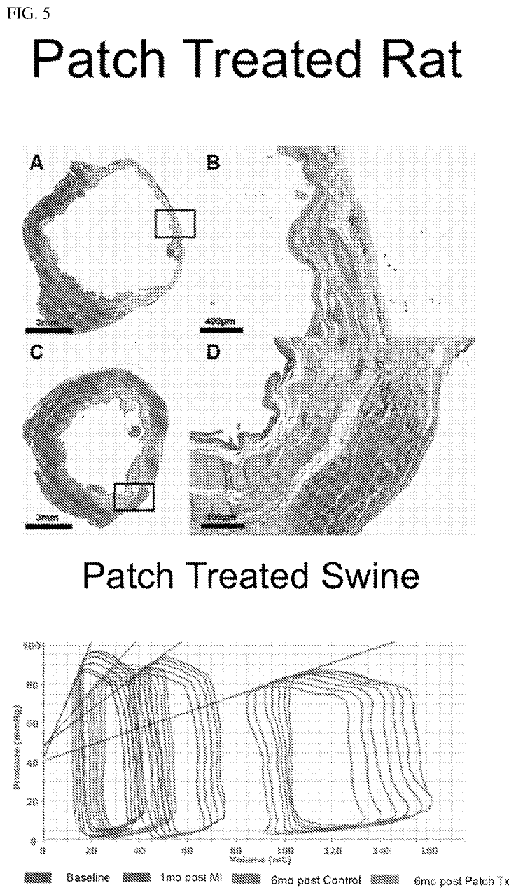

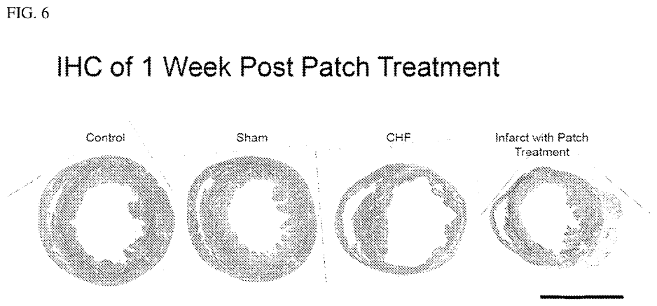

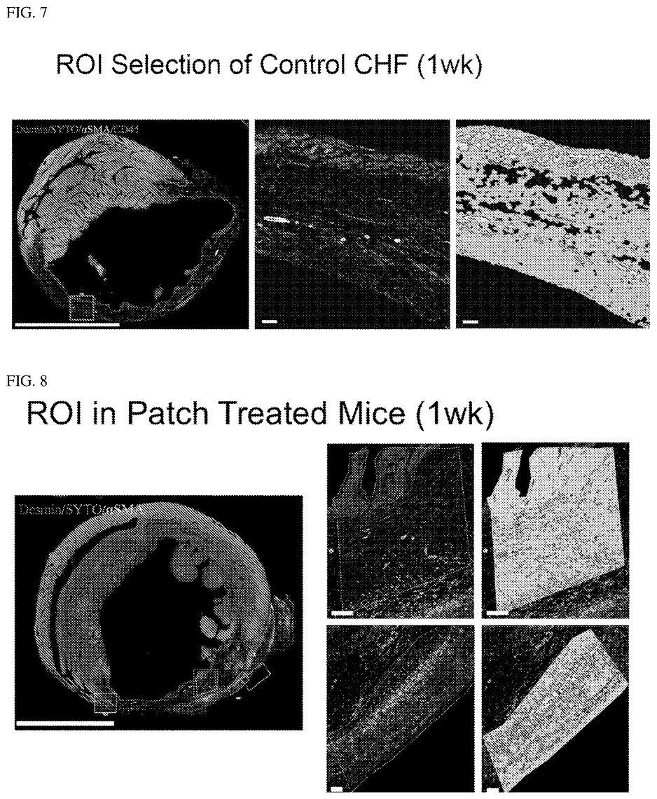

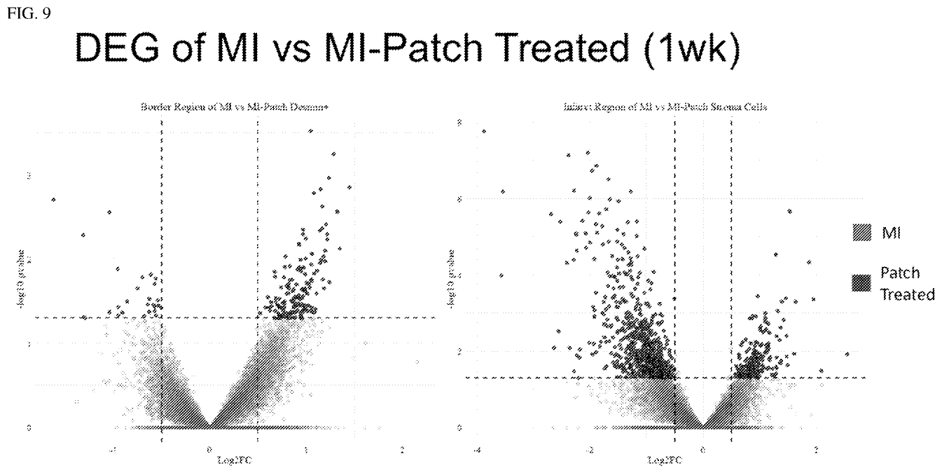

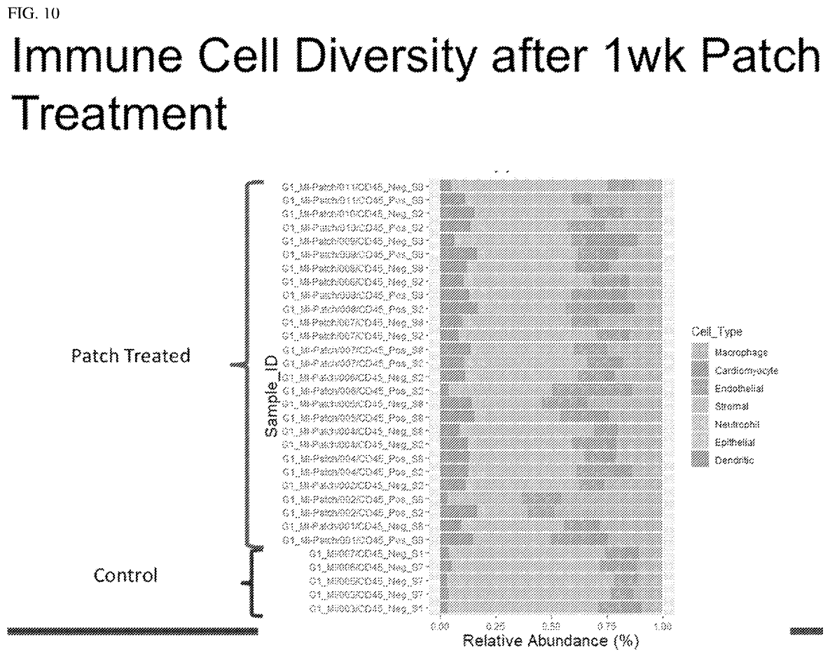

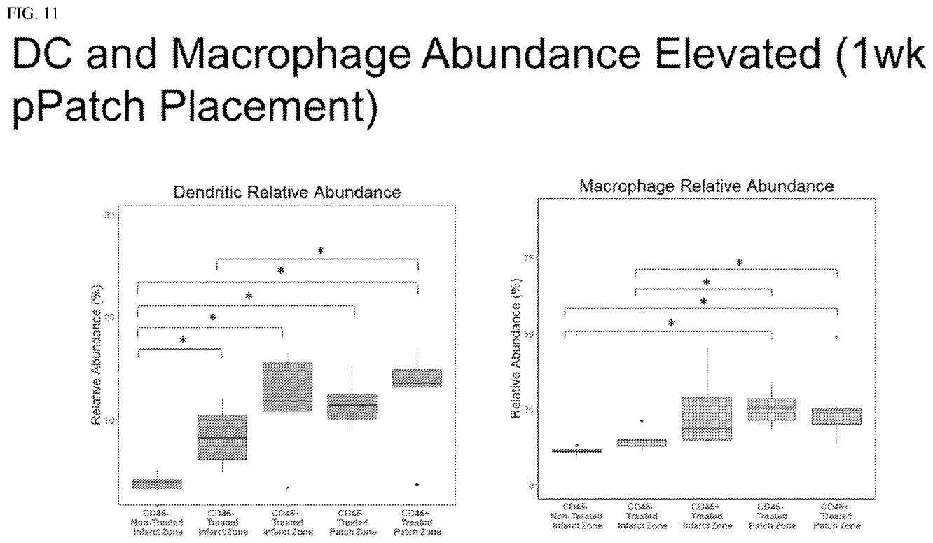

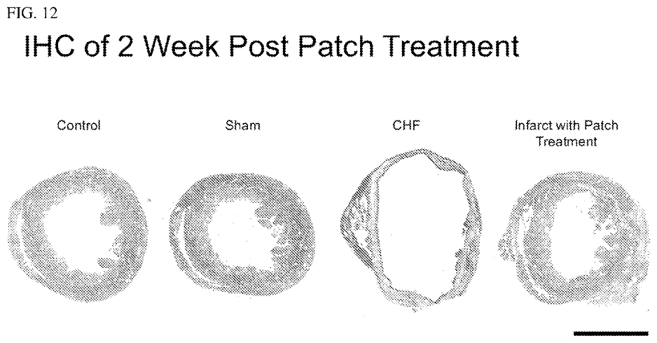

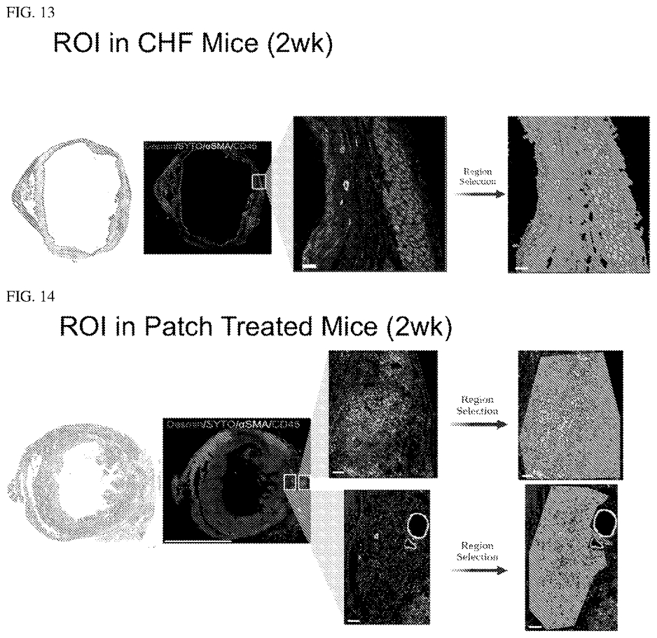

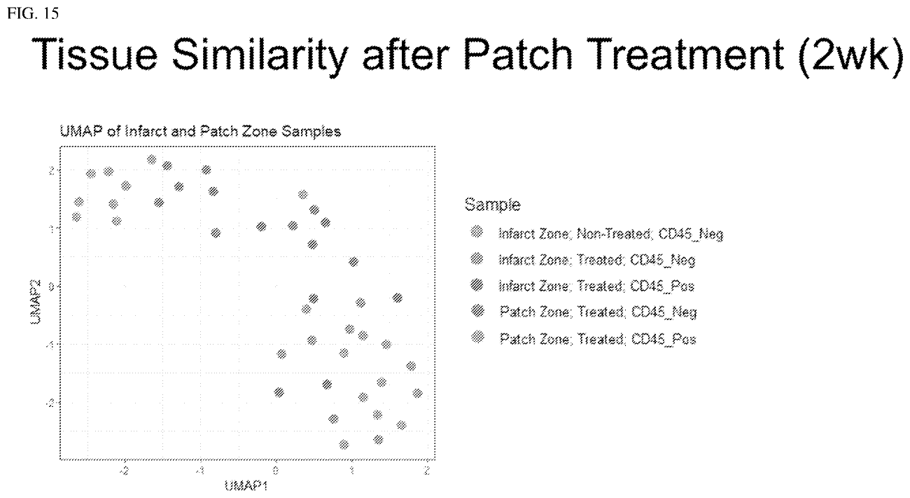

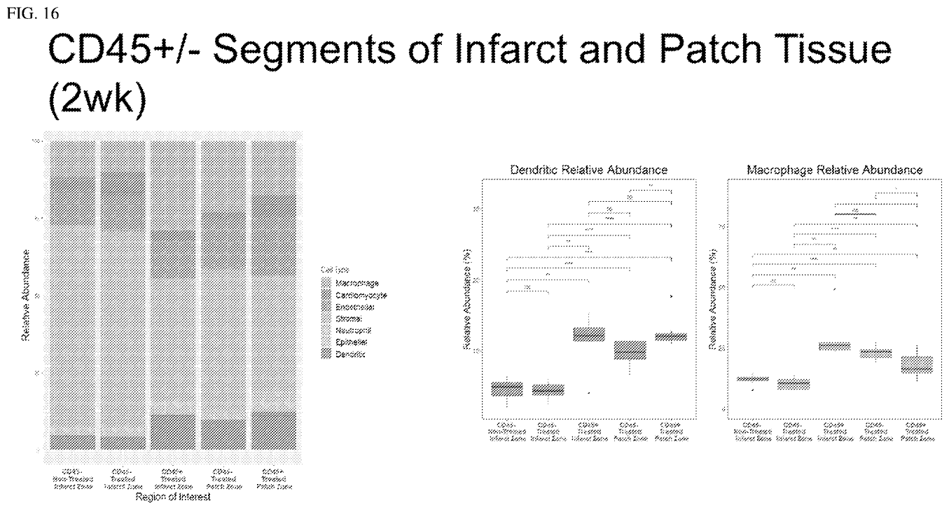

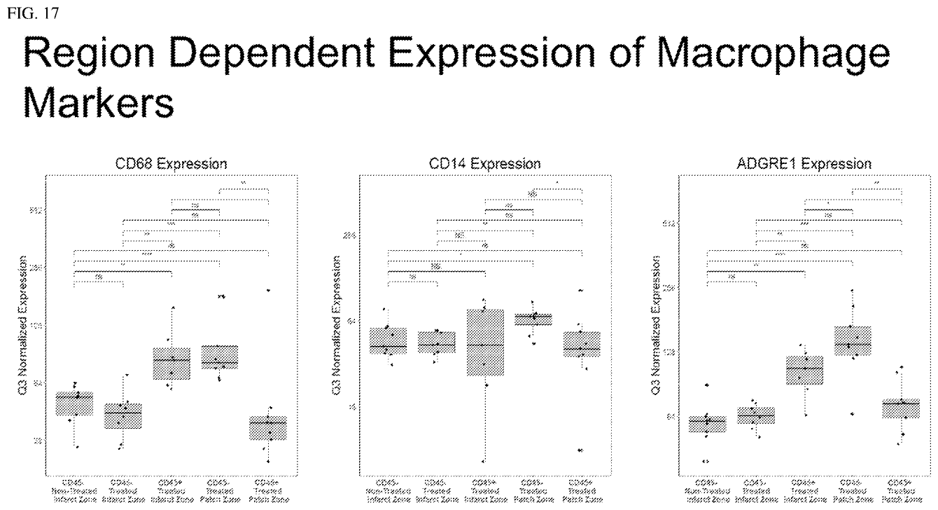

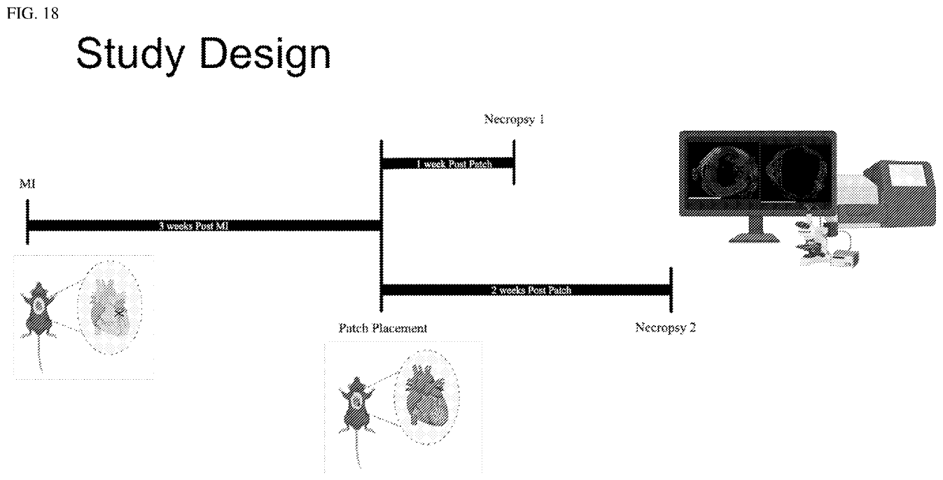

In summary, macrophage abundance was confirmed via macrophage markers including CD68, CD14, and ADGRE. Macrophage polarization was determined by M2 specific markers such as RETNLA, ARG1, and CHIL3 expression. Upregulation of M2 phenotypes such as FN1 and MRC1 was observed. The patch Induces differential expression of genes after one week of treatment. The experiments identified host innate and adaptive immune system implicated in cardiac regeneration. Recruitment/Activation of M2 macrophages may attenuate cardiac remodeling. Additional results are shown in FIGS. 3-18.

### Example 2

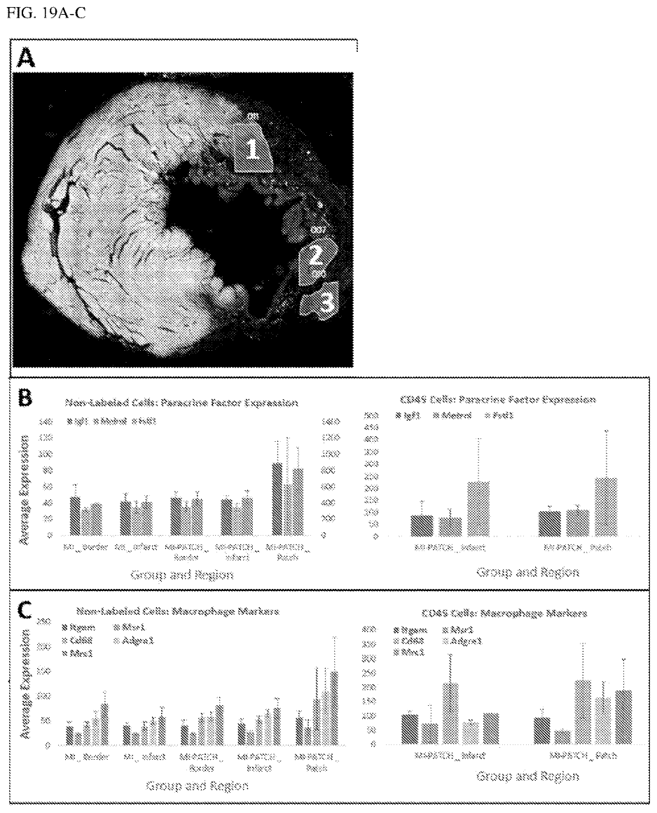

In the murine model of CHF, DSP obtained whole transcriptome analyses of three cell groups: 1) cardiomyocytes, 2) CD45 positive cells, and 3) unlabeled cells (FIG. 19A). Unlabeled cells were stained with a nuclei stain, however, had no other bio-markers due to technical limitations. This study revealed the iPSC-CMs, and fibroblasts recruited a substantial number of immune cells that express regenerative cytokines associated with angiogenesis and cardiomyocyte survival such as IGF1, Fstl1, and Metrnl (FIG. 19B). This immune response that grossly lower at similar timepoints in MI only mice. The cell types present in the CD45+ and unlabeled cell sections were examined and considerable expression of monocyte derived and tissue resident (TR) macrophage markers including CD68, Adgre1, and Itgam was found (FIG. 19C). These markers are highly upregulated in the CD45+ tissue sections, and the unlabeled cells within the patch (MIPatch_Patch) in treated mice compared to untreated MI mice. These markers were not upregulated in the border zone or infarct area (FIG. 19C). Transcripts obtained from CD45+ and unlabeled cell sections are highly enriched for M2/TR markers such as Msr1 and Mrc1 (FIG. 19C). This indicates that the patch bolsters an immune response, recruiting and expanding reparative macrophages.

### Example 3

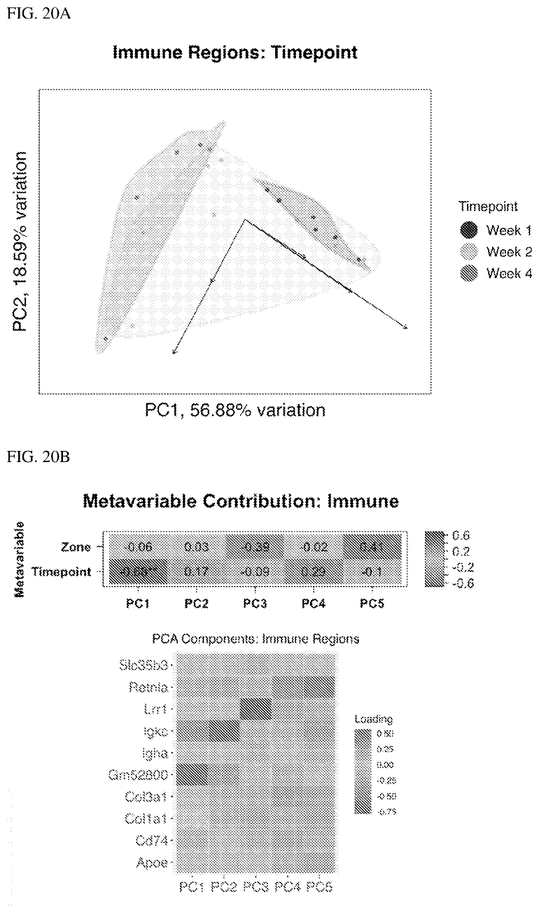

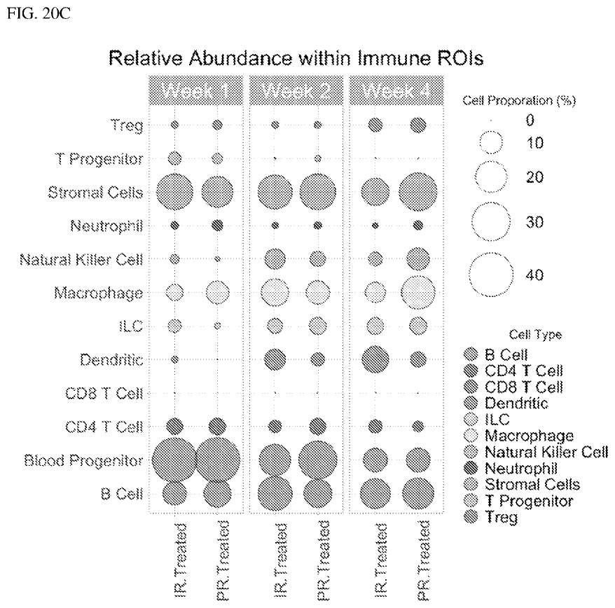

Additional transcriptomic experiments were conducted to identify drivers of immune modulation. FIG. 20 shows that the immune response was only present in patch-treated animals. Principal component 1 (PC1) is negatively correlated with timepoint and identifies a dynamic immune response over a time course (FIG. 20A). To identify the genes correlated with PC1 a heat map was generated (FIG. 20B). The heat map shows that Gm52800 gene, an uncharacterized protein associated with myeloid based immune responses positively correlated with time. Igkc is a B cell specific transcript. CD74 is a MHCII component. Retnla is a tissue protective, anti-inflammatory protein shown to be expressed by B cells. Relative abundance of immune cells within the Immune ROIs identifies, a reduction a recruitment in blood progenitor cells over time and an expanding population of Treg cells (FIG. 20C).

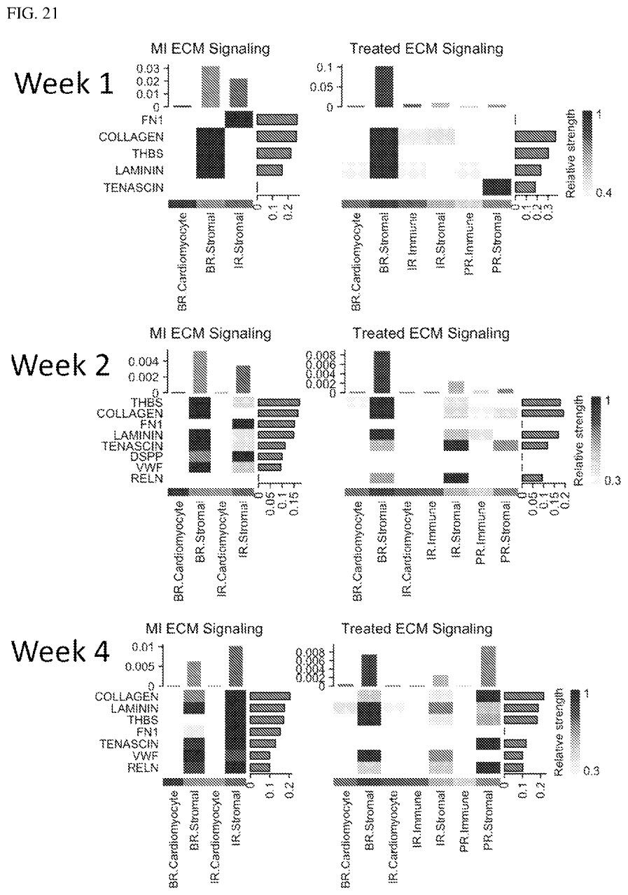

FIG. 21 shows a time course of extracellular matrix (ECM) cell signaling after patch placement. The patch induced endogenous changes in expression of collagen, laminin, THBS and fibronectin in border/patch region stromal cells of treated hearts. Immune and patch cells contribute to positive remodeling of the ventricle as shown in the time course:

### Week 1:

The stromal border region of MI and MI treated hearts plays a role in outgoing ECM signaling. This is highlighted by the increase in collagen, laminin, THBS in week 1 and contributes to pathologic fibrosis. TNC is highly expressed in the patch stromal region which may influence and dampen the immune response.

### Week 2:

Ths e repsone from week one is carried into week two in MI alone groups. In MI patch treated anti-inflammatory signaling originating from the infarct stromal regions via elevated expression of RELN and TNC.

### Week 4:

Pathologic fibrosis of MI alone tissues is further identified in week 4 via elevated expression across all collagen depositing proteins. This response is largely absent in the MI treated with continued elevated expression of anti-inflammatory transcripts and limited out going signaling for fibrosis compared to the MI alone. Reduction of adverse ECM signaling associated with heart failure is downregulated over the time course and is appreciated in week 4

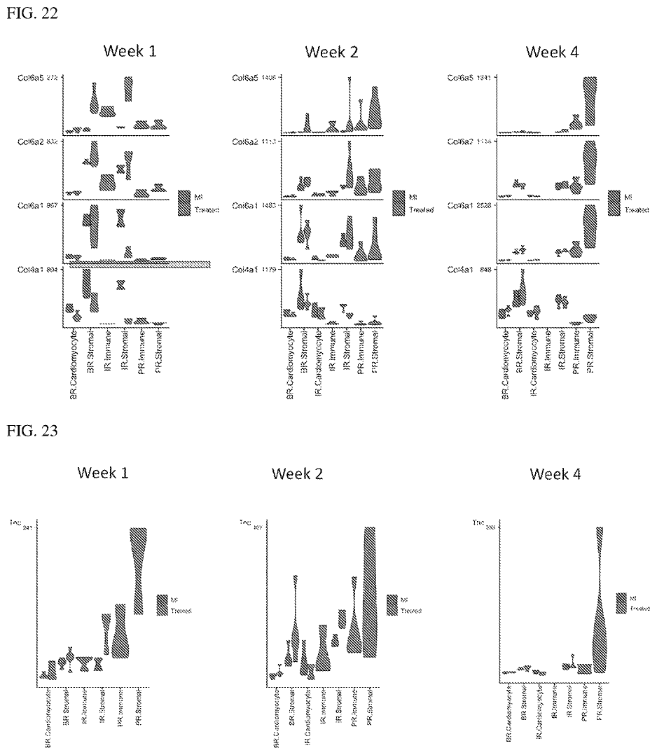

FIG. 22 shows a time course of specific collagen fiber types assessed at all time points. Over time increased expression of Col6a5 is observed with response to treatment. Week 4 demonstrates Col6 fiber subtypes over time become more expressed in the patch region stromal cells. This indicates the patch as a source of ECM signaling and remodeling that attenuates adverse remodeling.

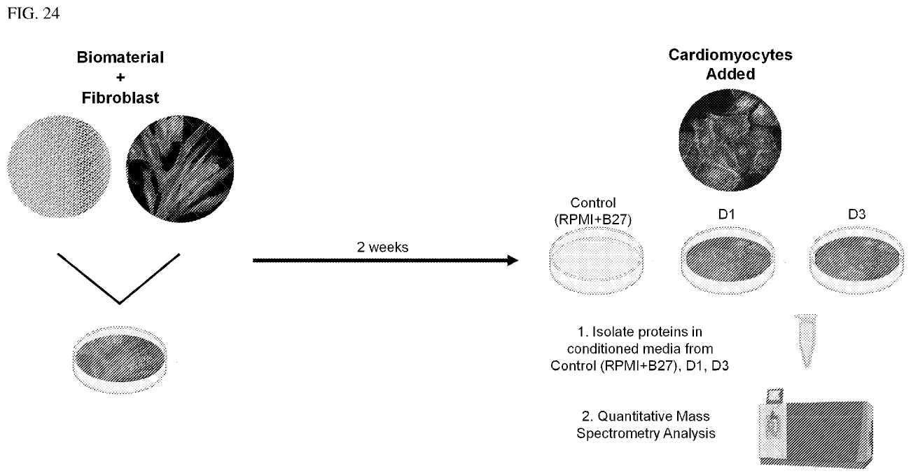

FIG. 23 shows a time course of Tensacin C (TNC) expression. TNC is a direct signaling pathway from the patch to myocardium. Elevated TNC expression in patch stromal cells plays a role in ECM interactions and modulation of the immune response. TNC is involved in epithelial/mesenchymal cell transition (EMT) and mesenchymal/epithelial transition (MET). TNC is involved in (1) the differentiation of cardiomyocytes from the mesoderm, (2) cushion tissue and valve formation, and (3) coronary vascular development.

Proteomic analysis of the patch secretome during manufacture is enriched for anti-inflammatory mechanism via regulation of ECM organization combined with M2 polarization signaled by IL-4 and IL-13. GO terms enriched for biological processes increase wound healing mechanisms, angiogenesis, and IGF signaling. These mechanisms are induced by the biologic

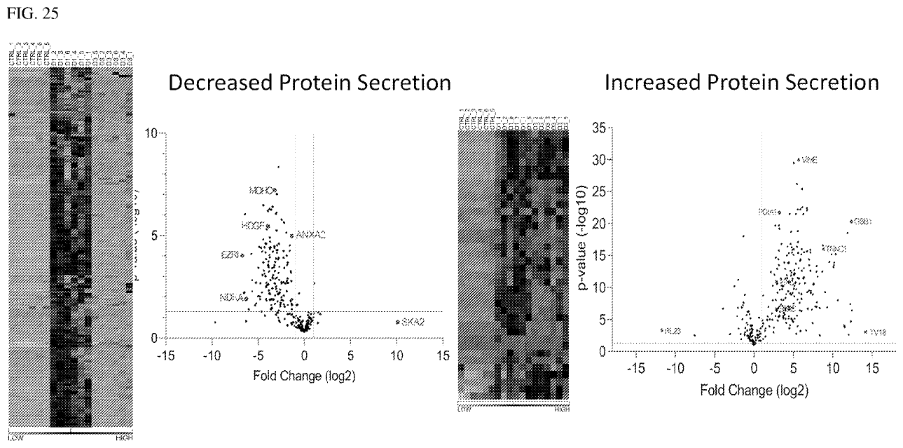

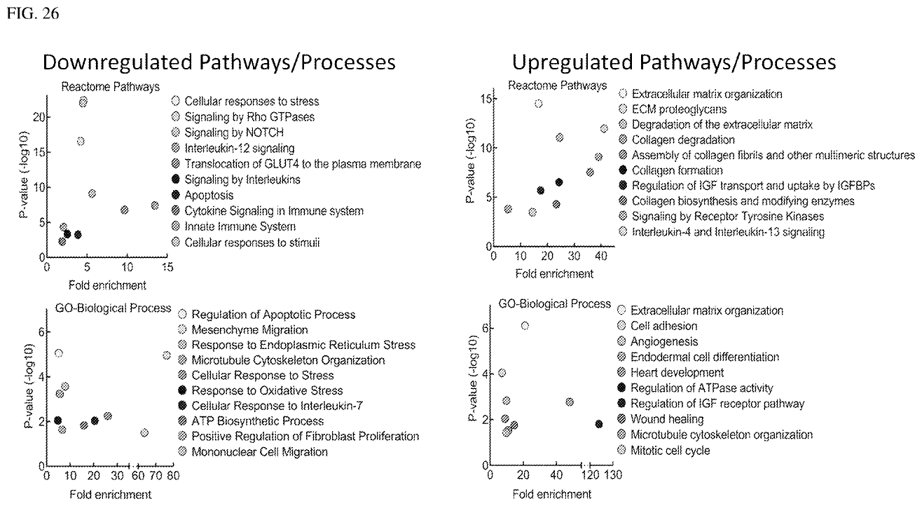

The patch proteome undergoes a biological shift in protein expression after addition of cardiomyocytes during the manufacture of MyCardia (FIG. 24). FIG. 25 shows two distinct proteomic profile and associated signaling pathways (FIG. 26). MyCardia established a unique proteomic secretion profile resulting in the down regulation of 157 proteins known to support M1 like macrophage polarization such as IL-12 and NOTCH and up regulation of 191 unique proteins associated with M2 like, pro-reparative polarization such as IL-4, IL 13 and signaling of IGF.

Tables 1 and 2 shows proteins up and down regulated after patch placement.

TABLE 1
Proteins Downregulated

| | | | | |
|---|---|---|---|---|
| 1433B_HUMAN | CAP1_HUMAN, | HS71L_HUMAN, | PDIA6_HUMAN, | RTN4_HUMAN, |
| 1433E_HUMAN | CAPZB_HUMAN, | HS90A_HUMAN, | PDIA6_HUMAN, | S10A6_HUMAN, |
| 1433T_HUMAN, | CAVN1_HUMAN, | HS90A_HUMAN, | PEA15_HUMAN, | SAP_HUMAN, |
| 1433Z_HUMAN, | CAZA1_HUMAN, | HS90B_HUMAN, | PGAM1_HUMAN, | SODM_HUMAN, |
| ACTA_HUMAN, | CH10_HUMAN, | HSP72_HUMAN, | POTEE_HUMAN, | SPTB2_HUMAN, |
| ACTB_HUMAN, | CLH1_HUMAN, | HSP7C_HUMAN, | PPIA_HUMAN, | SPTN1_HUMAN, |
| ACTC_HUMAN, | COF1_HUMAN, | IF4A2_HUMAN, | PRDX1_HUMAN, | TBB1_HUMAN, |
| ACTH_HUMAN, | DX39B HUMAN, | KPYM HUMAN, | PRDX1_HUMAN, | TBB1_HUMAN, |
| ACTN1_HUMAN, | EF1A1_HUMAN, | KPYR_HUMAN, | PRDX2_HUMAN | TBB2A_HUMAN, |
| ACTN2_HUMAN, | EF2_HUMAN, | KT_HUMAN, | PRDX4_HUMAN, | TBB2A_HUMAN, |
| ACTN3_HUMAN, | ENPL_HUMAN, | LAMP1_HUMAN, | PRDX5_HUMAN, | TBB3_HUMAN, |
| ACTN4_HUMAN | ENPL_HUMAN, | LAMP2_HUMAN, | PRDX6_HUMAN, | TBB3_HUMAN, |
| ACTS_HUMAN | ERO1A_HUMAN, | LEG1_HUMAN, | PROF1_HUMAN, | TBB4A_HUMAN, |
| ALDOA_HUMAN, | FLNA_HUMAN, | LEG3_HUMAN, | PSA4_HUMAN, | TBB4A_HUMAN, |
| ALDOC_HUMAN, | FLNB_HUMAN, | LMNA_HUMAN, | PSB2_HUMAN, | TBB5_HUMAN |
| AMPN_HUMAN, | G3P_HUMAN, | LMNB1_HUMAN, | RL12_HUMAN, | TBB5_HUMAN, |
| ANXA1_HUMAN, | G6PI_HUMAN, | LMNB1_HUMAN, | RLA2_HUMAN, | TBB6_HUMAN, |
| ANXA2_HUMAN | GLU2B_HUMAN, | MIF_HUMAN, | ROA2_HUMAN, | TBB6_HUMAN, |
| ANXA2_HUMAN, | GSTP1_HUMAN, | MOES_HUMAN, | RS10_HUMAN, | TBB8B_HUMAN, |
| ATPA_HUMAN | H2B1A_HUMAN | NDKA_HUMAN, | RS16_HUMAN, | TCPH_HUMAN, |
| BIP_HUMAN, | H2B1A_HUMAN, | PDIA1_HUMAN, | RS18_HUMAN, | TERA_HUMAN, |
| CALM1_HUMAN, | H2B3B_HUMAN, | PDIA1_HUMAN, | RS27A_HUMAN, | TERA_HUMAN, |
| CALR_HUMAN, | H4_HUMAN, | PDIA3_HUMAN, | RS8_HUMAN, | TKT_HUMAN, |
| CALR_HUMAN, | HDGF_HUMAN, | PDIA4_HUMAN | RSSA_HUMAN, | TPM3_HUMAN, |
| | | | | TXND5_HUMAN, |
| | | | | UTS2_HUMAN, |
| | | | | VIME_HUMAN, |

TABLE 2
Proteins Upregulated

MMP3
FBLN2
CO1A1
CO1A2
BGH3
PTX3
MMP2
CO6A3
Co6A1
FINC
THY1
CO6A2
TSp1
SPRC
MYG
TPM2
TNNC1
IBP4
IPB2
PGS2
TBA1A
TBA3C
TBA3E
PGS1
LUM
CATB
TIMP1
CYTC
APOE

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A method of repairing a tissue, comprising
contacting said tissue with a composition comprising one or more polypeptides and/or proteins selected from the group consisting of those shown in Table 2.

2. A method of repairing a tissue, comprising
contacting said tissue with a composition comprising a nucleic acid encoding one or more polypeptides and/or proteins selected from the group consisting of those shown in Table 2.

3. A method of repairing a tissue, comprising
contacting said tissue with a composition that induces or decreases expression of genes associated with tissue repair and/or regeneration of said tissue.

4. The method of claim 3, wherein said composition induces differential expression of genes involved in host innate and adaptive immune system and/or induces recruitment/activation of M2 macrophages, M1 macrophages, and/or stromal cells.

5. The method of claim 4, wherein said genes are one or more of RETNLA, ARG1, CHIL3, FN1, and MRC1.

6. The method of any of the preceding claims, wherein said composition comprises cells that express one or more exogenous genes selected from the group consisting of RETNLA, ARG1, CHIL3, FN1, and MRC1.

7. The method of any of the preceding claims, wherein said composition decreases expression of one or more genes selected from those shown in Table 1 and increases expression of one or more genes shown in Table 2.

8. The method of any of the preceding claims, wherein said composition regulates ECM organization.

9. The method of any of the preceding claims, wherein said composition promotes signaling by one or more molecules selected from the group consisting of IL-4, IGF, and IL-13.

10. The method of any of the preceding claims, wherein said tissue is a muscle.

11. The method of claim 10, wherein said muscle is heart muscle or skeletal muscle.

12. The method of any of the preceding claims, wherein said tissue is skin, heart, kidney, bone marrow, brain, liver, or lung.

13. The method of any of the preceding claims, wherein said composition reduces fibrosis and/or inflammation in said tissue.

14. The method of any of the preceding claims, wherein said composition increases fibrosis and/or inflammation in said tissue.

15. The method of any of the preceding claims, wherein said contacting treat a disease or disorder.

16. The method of claim 15, wherein said disease or disorder is selected from the group consisting of fibrosis, blast wave injury, muscle loss, skin damage, CHF, and aging.

17. The method of any of the preceding claims, wherein said composition comprises a solid or semi-solid support.

18. The method of claim 17, wherein said solid or semi-solid support is selected from the group consisting of a scaffold, a gel, a nanoparticle, or a patch.

19. The method of claim 18, wherein said polypeptide, proteins, or nucleic acid is affixed to said solid or semi-solid support.

20. The method of any of the preceding claims, wherein said composition comprises a pharmaceutically acceptable carrier.

21. The method of any of the preceding claims, wherein said composition comprises a virus-like particle encoding said one or more polypeptides and/or proteins.

22. A composition, comprising a solid or semi solid support comprising one or more polypeptides and/or proteins selected from the group consisting of those shown in Table 2.
